(19) United States
(12) Patent Application Publication
(10) Pub. No.: US 2009/0030246 A1
(43) Pub. Date: Jan. 29, 2009

(54) PROCESS FOR FLUORINATION USING 1,1,2,2-TETRAFLUOROETHYL-N,N-DIMETHYLAMINE

(75) Inventors: Christopher P. JUNK, Wilmington, DE (US); Viacheslav A. Petrov, Hockessin, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. du Pont de Nemours and Company, Wilmington, DE (US)

(21) Appl. No.: 12/178,873

(22) Filed: Jul. 24, 2008

Related U.S. Application Data
(60) Provisional application No. 60/962,240, filed on Jul. 27, 2007.

(51) Int. Cl. C07C 17/26

(52) U.S. Cl. 570/142

(57) ABSTRACT

A process for making a fluorinated product comprising contacting an alcohol with 1,1,2,2-tetrafluoroethyl-N,N-dimethylamine to produce a product mixture containing the fluorinated product and N,N-dimethyl difluoroacetamide, quenching the product mixture in water to form a first organic phase and an aqueous phase, recovering fluorinated product by separating the first organic phase from the aqueous phase, treating the aqueous phase to recover N,N-dimethyl difluoroacetamide, and converting recovered N,N-dimethyl difluoroacetamide to 1,1,2,2-tetrafluoroethyl-N,N-dimethylamine.

### BACKGROUND OF THE INVENTION

The present invention relates to a process for making fluorinated product and more specifically relates to making a fluorinated product from a feed alcohol using 1,1,2,2-tetrafluoroethyl-N,N-dimethylamine as a fluorinating agent and regenerating the fluorinating agent for reuse.

The conversion of an alcohol to the corresponding fluoride is conventionally done using the commercially available fluorinating agent, 1,1,2,3,3,3-hexafluoropropyl-N,N-diethyl amine. The reaction is:

ROH+CF₃CFHCF₂—N(C₂H₅)₂→RF+CF₃CFHCO—N(C₂H₅)₂+HF

R represents any organic group, such as substituted or unsubstituted alkyl or arylalkyl. The reaction may be run neat (without solvent), or in a solvent such as methylene chloride, chloroform, carbon tetrachloride, or ethyl ether. To recover the product, the product mixture is contacted with cold (or ice) water and the organic phase separated. This phase contains the product fluoride and much of the amide derived from 1,1,2,3,3,3-hexafluoropropyl-N,N-diethyl amine. The product fluoride must be separated, usually by distillation. Such distillation can be difficult in cases where the product fluoride and byproduct amide have similar boiling points, or where the product fluoride is heat sensitive. In addition, there is no convenient method for regeneration of 1,1,2,3,3,3-hexafluoropropyl-N,N-diethyl amine from the amide byproduct.

There is a need for an improved process for fluorination, particularly in applications such as pharmaceutical and agricultural manufacturing, where the large scale of the processes place a premium on simple separation methods that give products in good yield and high purity with a minimum of waste generation, for example by regenerating the fluorinating agent after fluorination.

### SUMMARY OF THE INVENTION

It has been discovered that by using 1,1,2,2-tetrafluoroethyl-N,N-dimethylamine as a fluorinating agent for a feed alcohol, a fluorinated product can be made and the 1,1,2,2-tetrafluoroethyl-N,N-dimethylamine regenerated for reuse. The fluorination reaction is carried out by contacting the feed alcohol with 1,1,2,2-tetrafluoroethyl-N,N-dimethylamine to produce a produce mixture containing the fluorinated product and a byproduct amide from 1,1,2,2-tetrafluoroethyl-N,N-dimethylamine which is N,N-dimethyl difluoroacetamide. The reaction is illustrated below:

ROH+HCF₂CF₂—N(CH₃)₂→RF+HCF₂CO—N(CH₃)₂+HF

wherein R represents any organic group, such as alkyl or arylalkyl, and the group may be substituted with fluoride, iodide, and/or bromide. The group may contain ether functionality and/or tertiary amine functionality. Incompatible substituents are acids, such as carboxylic and sulfonic acids. As used in the present application, the terms feed alcohol and fluorinated product, represented by ROH and RF in the above reaction, encompass the variations described above for the organic group R.

The product mixture is quenched in water to produce a first organic phase and an aqueous phase. Fluorinated product is recovered by separating the first organic phase from the aqueous phase. Preferably, the aqueous phase is extracted with a liquid hydrocarbon to form a second organic phase and an extracted aqueous phase, the second organic phase is combined with the first organic phase and the liquid hydrocarbon is evaporated from the combined organic phase to recover the fluorinated compound. The aqueous phase containing the byproduct amide is treated to recover the amide and the recovered amide is converted back to 1,1,2,2-tetrafluoroethyl-N,N-dimethylamine.

The process can produce the fluorinated product in good yield and high purity and avoids the need to separate the fluorinated product and the byproduct amide by distillation. Moreover, the amide byproduct is converted back to the 1,1,2,2-tetrafluoroethyl-N,N-dimethylamine fluorinating agent. Thus, the present invention can provide fluorinated products in good yield and high purity with a minimum of waste generation.

### DETAILED DESCRIPTION OF THE INVENTION

1,1,2,2-tetrafluoroethyl-N,N-dimethylamine is made by reaction of dimethylamine (DMA) with tetrafluoroethylene (TFE). 1,1,2,2-Tetrafluoroethyl-N,N-dimethylamine is a low-viscosity liquid (boiling point 32° C. at 127 mm Hg (17 kPa)). It hydrolyzes readily in moist air and reacts vigorously with water and alcohols, so it must be kept dry to maintain its activity as a fluorinating agent. The chemistry of 1,1,2,2-tetrafluoroethyl-N,N-dimethylamine is discussed in V. Petrov, et al., J. Fluorine Chem., vol. 109, pp. 25-31 [2001].

The reaction of 1, 1,2,2-tetrafluoroethyl-N,N-dimethylamine with a feed alcohol to make an fluorinated product should be conducted in a vessel whose materials of construction are resistant to attack by hydrogen fluoride (HF), a product of the reaction. In laboratory-scale reactions fluoropolymer vessels are suitable, such as those made of tetrafluoroethylene/perfluoro(alkyl vinyl ether) copolymers sold under the trademark Teflon® PFA. The 1,1,2,2-tetrafluoroethyl-N,N-dimethylamine is preferably added to the vessel first. It may be used neat, or in a solvent such as methylene chloride or chloroform. Depending upon the feed alcohol and the reactor employed, the reaction can be carried out at a temperature range from about −50° C. to about 100° C. Preferably, the vessel and contents are cooled to below 0° C. prior to addition of 1,1,2,2-tetrafluoroethyl-N,N-dimethylamine, more preferably to about −10° to −25° C. In laboratory-scale reactions, dry ice-acetone mixture is a convenient cooling medium, capable of cooling to about −78° C. The reactant alcohol may be added dropwise to the stirred cold 1,1,2,2-tetrafluoroethyl-N,N-dimethylamine at a rate that does not cause a rise in reaction temperature to greater than about 0° C. After addition is complete, cooling may be removed and the reaction allowed to proceed at from about room temperature (20-25° C.) to about 50° to 75° C. Reaction time can range from about 1 to about 48 hours, preferably from about 4 to 24 hours.

The feed alcohol should be dry, i.e. preferably contain less about than 0.1 wt % water, more preferably less than about 0.05 wt % water. The reaction vessel should also be dry and flushed with dry nitrogen to avoid introduction of moisture in the air. The presence of small amounts of water will not prevent the reaction of 1,1,2,2-tetrafluoroethyl-N,N-dimethylamine with alcohol, but the water will react with 1,1,2,2-tetrafluoroethyl-N,N-dimethylamine, resulting in a waste of 1,1,2,2-tetrafluoroethyl-N,N-dimethylamine. One mole of 1,1,2,2-tetrafluoroethyl-N,N-dimethylamine will convert one mole of alcohol to the corresponding fluoride, so at least a stoichiometric amount of 1,1,2,2-tetrafluoroethyl-N,N-dimethylamine should be used, i.e. one mole 1,1,2,2-tetrafluoroethyl-N,N-dimethylamine per mole of feed alcohol. Preferably an excess of 1,1,2,2-tetrafluoroethyl-N,N-dimethylamine is used to allow for traces of water in the alcohol, moisture in the reaction vessel and moisture introduced otherwise. Preferably the ratio of 1,1,2,2-tetrafluoroethyl-N,N-dimethylamine:feed alcohol is at least about 1.1. There is no harm in having an excess of 1,1,2,2-tetrafluoroethyl-N,N-dimethylamine, but for economy, the ratio preferably is no greater than about 1.25.

To begin recovery of the fluorinated product, if the reaction has been conducted in solvent, the product mixture may be subjected to reduced pressure to strip off the solvent. The product mixture is then quenched in water such as by pouring over cold water or ice. Preferably, the amount of water is at least about 20% the weight of the reaction mixture and may be employed in quantities of up to 10 times the weight of the reaction mixture. The water will hydrolyze remaining 1,1,2,2-tetrafluoroethyl-N,N-dimethylamine and absorb the heat generated thereby. Preferably, the reaction mixture is poured into water containing acid such as cold hydrochloric acid (10 wt %). It is preferable for the water to contain acid since the acid increases the solubility of byproduct amide the aqueous phase, and decreases solubility of the fluorinated product. In larger-scale reactions, the reaction mixture can be quenched by controlled addition to well-stirred cold water containing HCl in a cooled vessel to ensure dissipation of heat of hydrolysis. Suitable temperatures can range from about 0° C. to about 50° C. The resulting mixture separates into two phases, one, the organic phase (referred to herein as the first organic phase, to distinguish it from the hydrocarbon extracts described below) being the fluorinated product. The other phase, an aqueous phase, contains most of the byproduct amide (N,N-dimethyl difluoroacetamide). The separation is clean giving the fluorinated product in good yield and high purity. This separation works particularly well when the fluorinated product is liquid, and better still, a low viscosity liquid (i.e., no more than ten times the viscosity of water).

If the fluorinated product is a solid, or a high viscosity liquid, or otherwise is present in significant quantity in the aqueous phase, it is preferred to extract the aqueous phase. In doing this, the first organic phase is set aside, and the aqueous phase is extracted at least once, and preferably several times with liquid hydrocarbon such as alkane or aromatic. Preferred alkanes are pentanes, hexanes, heptanes, and octanes, and mixtures thereof. Preferred aromatic hydrocarbons are benzene, toluene, and the xylenes. The hydrocarbon extracts and the first organic phase are preferably combined and then preferably washed at least once with cold water to remove any traces of N,N-dimethyl difluoroacetamide in the extracts and first organic phase. The byproduct amide, N,N-dimethyl difluoroacetamide, partitions essentially completely into the aqueous phase, so the extraction leaves the fluorinated product in the first organic phase and the hydrocarbon extract(s) and the N,N-dimethyl difluoroacetamide by product in the aqueous phase. The hydrocarbon extract(s), preferably combined with the first organic phase, may then be easily concentrated by evaporation to remove the hydrocarbon and, if used, any reaction solvent. If evaporation of the hydrocarbon extract(s) and of the first organic phase are done separately, then the products of evaporation can be combined to provide the fluorinated product. Similarly, if only the hydrocarbon extracts are evaporated, the product of evaporation and the fluorinated product separated from the aqueous phase can be combined. The combined fluorinated product produced is typically very pure fluorinated product. Purity is typically greater than 90%, more often greater than 95%. Yields (based on the feed alcohol) typically exceed 60%.

If the above-described procedure of the invention is practiced with the prior art fluorinating agent 1,1,2,3,3,3-hexafluoropropyl-N,N-diethyl amine instead of 1,1,2,2-tetrafluoroethyl-N,N-dimethylamine, much of the byproduct amide, N,N-diethyl 1,2,2,2-tetrafluoropropionamide remains in the organic phase and cannot be separated from the fluorinated product by extraction. Therefore, obtaining the fluorinated product in high purity requires further purification such as by distillation, which because of the high-boiling nature of the byproduct amide, can be difficult and in any case results in reduced yield and increased cost.

In the process of the present invention, the clean separation of the fluorinated product from the byproduct amide additionally results in the aqueous phase containing the more than 90% of the byproduct amide contaminated only by unreacted alcohol starting material. Preferably, HCF₂C(O)N(CH₃)₂ is separated by treatment with weak base (aqueous NaHCO₃ or K₂CO₃) to remove HF, followed by simple distillation to recover the byproduct amide. Then, the amide is reacted to convert it back to the starting material fluorinating agent, 1,1,2,2-tetrafluoroethyl-N,N-dimethylamine. The conversion is preferably done according to the procedure for the reaction of dimethyl formamide (HC(O)N(CH₃)₂) with COF₂, producing HCF₂N(CH₃)₂ (Experimental, Section E. at p. 4281 in F. S. Fawcett, et al., J. Am. Chem. Soc, (1962) v. 84, pp. 4275-85). The reaction proceeds in one step by exposure of the amide to carbonyl fluoride at 20-150° C. for 2-24 hr:

HCF₂C(O)N(CH₃)₂+COF₂→HCF₂CF₂N(CH₃)₂

Alternatively, a two-step method is advantageously used, the first step being reaction with conventional chlorinating agents (COCl₂, oxalyl chloride, or diphosgene, followed by the second step, reaction of resulting dichloride with alkali metal fluoride or tetraalkylammonium fluoride in polar solvent to produce the corresponding difluoride. This reaction is disclosed in U.S. Pat. No. 6,329,529 at column 15, line 27 bridging to column 16, line 30. Although the description is for conversion of ureas, the procedure applies to HCF₂C(O)N(CH₃)₂ as well. The recovery scheme is as follows: Step 1: The reaction of the amide with phosgene or oxalyl chloride to convert the amide to the dichloride:

HCF₂C(O)N(CH₃)₂+COCl₂→HCF₂CCl₂N(CH₃)₂

The reaction is run at 0-100° C. for 1-24 hr in polar solvent that is unaffected by the action of chlorinating agent (for example CH₃CN), Step 2: The dichloride is treated with a metal or alkyl ammonium fluoride or bifluoride, such as NaF, KF, CsF, R₄N⁺F⁻ in a polar solvent such as acetonitrile, dimethylformamide, glymes or HCF₂C(O)N(CH₃)₂ at 50-200° C. for 2-24 hr:

HCF₂CCl₂N(CH₃)₂+metal fluoride→HCF₂CF₂N(CH₃)₂

The product then need only be distilled to recover 1,1,2,2-tetrafluoroethyl-N,N-dimethylamine in purity of at least 90%, and usually greater than 95%. In either the one step or two step methods, the conversion proceeds in good yield, typically exceeding 70% and more usually, 85%.

### EXAMPLES

### Example 1

### Reaction of (S)-methyl mandelate with 1,1,2,2-Tetrafluoroethyl-N,N-dimethylamine

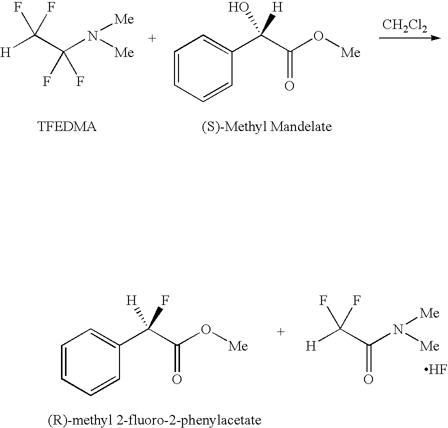

This optically active mandelate demonstrates the stereospecificity of the reaction with 1,1,2,2-tetrafluoroethyl-N,N-dimethylamine. 1,1,2,2-tetrafluoroethyl-N,N-dimethylamine (8.5 g, 59 mmol) is added to a 50 mL fluoropolymer (Teflon® PFA) flask equipped with pressure equalized addition funnel. A digital thermometer is inserted and flask the flask cooled under positive N₂ pressure in dry ice/acetone bath to −20° C. with magnetic stirring. In a separate flask, (S)-methyl mandelate (Sigma Aldrich, >95%, 6.5 g, 39 mmol) is combined with 50 mL dry CH₂Cl₂ and 0.5 g MgSO₄ to ensure dryness. (S)-methyl mandelate solution is filtered using a 0.45 μm syringe filter into the addition funnel. (S)-methyl mandelate solution is added dropwise to the cooled 1,1,2,2-tetrafluoroethyl-N,N-dimethylamine reagent with stirring. The bath is removed and the solution is allowed to warm to room temperature (RT) and stirred for 4 hr.

The crude reaction contents are transferred to a 100 mL round bottom flask for distillation. Methylene chloride solvent is removed at RT under reduced pressure (20 torr (2.7 kPa)), leaving a mixture (13.6 g) of product and N,N-dimethyl difluoroacetamide. This mixture is slowly poured over 10 mL of cooled 10% aqueous HCl solution. No exotherm is observed during quenching. After this mixture warms to RT, the organic phase is removed and set aside. The aqueous phase is extracted 3 times with 10 mL of hexane (3×10 mL). The organic phase is combined with the hexane extracts and further washed with deionized water (3×2 mL). The organic phase is dried over MgSO₄ and concentrated in vacuo to afford 4.5 g of material which was characterized by ¹⁹F and ¹H NMR, and gas chromatography/mass spectroscopy.

### Product Analysis and Mass Balance:

Of the 4.5 g isolated material:
95 wt. % (4.3 g) is the desired product: methyl 2-fluoro-2-phenylacetate.
3 wt. % (0.1 g) is residual byproduct amide (N,N-dimethyl difluoroacetamide).

The aqueous phase and the deionized water washes are combined and treated with 25 ml of 5 wt % aqueous NaHCO₃ solution to neutralize acid. The resulting solution is distilled to isolate the N,N-dimethyl difluoroacetamide.
2 wt. % (0.1 g) is methyl mandelate dimer product.
   Of the 7.2 g expected N,N-dimethyl difluoroacetamide:
99 wt. % (7.1 g) was removed during water washes.
1 wt. % (0.1 g) stayed with the product.
   Of the 6.6 g expected (R)-methyl 2-fluoro-2-phenylacetate product:
65 wt. % (4.3 g) was recovered.

The formation of the fluoride results in an enantiomeric excess of 26 %, meaning a 67/33 ratio of inversion to retention product, respectively.
35 wt. % (2.3 g) was lost during water washes.

The reaction gives the product fluoride in high purity (95%) and good yield (65%).

### Example 2

### Reaction of Cyclohexylmethanol with 1,1,2,2-Tetrafluoroethyl-N,N-dimethylamine

The reaction of Example 1 is repeated with the replacement of methyl mandelate with cyclohexylmethanol (C₆H₁₁CH₂OH). The desired fluorinated product is cyclohexylmethyl fluoride (C₆H₁₁CH₂F). The conditions are generally those of Example 1 except that no solvent is used. The product mixture is poured over cold 10% aqueous HCl. An organic and an aqueous phase form. The organic phase is separated. Analysis shows it to be cyclohexylmethyl fluoride of >95% purity. The yield is 75% based on the starting alcohol. N,N-Dimethyl difluoroacetamide is recovered as described in Example 1.

### Comparative Example 1

### Reaction of (S)-methyl mandelate with 1,1,2,3,3,3-hexafluoropropyl-N,N-diethyl amine

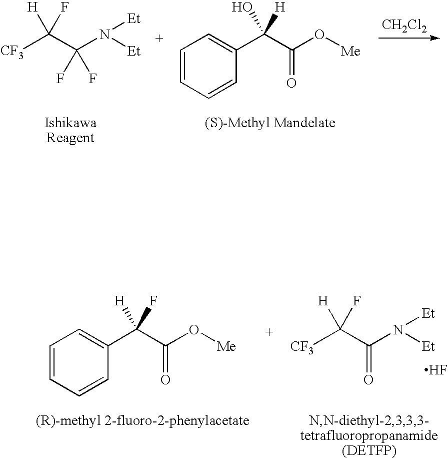

1,1,2,3,3,3-Hexafluoropropyl-N,N-diethyl amine (Ishikawa Reagent, TCI 95%, 10.1 g, 4.5 mmol) is added to a 50 mL fluoropolymer (Teflon® PFA) flask equipped with pressure equalized addition funnel. A digital thermometer is inserted and the flask cooled under positive N₂ pressure in dry ice/acetone bath to −20° C. with magnetic stirring. In a separate flask, (S)-methyl mandelate (Sigma Aldrich, >95%, 5.1 g, 3.1 mmol) is combined with 50 mL dry CH₂Cl₂ and 0.5 g MgSO₄ to ensure dryness. (S)-methyl mandelate solution is filtered through a 0.45 μm syringe filter into the addition funnel. (S)-methyl mandelate solution is added dropwise to the cooled 1,1,2,3,3,3-hexafluoropropyl-N,N-diethyl amine with stirring. The bath is removed and the solution is allowed to warm to room temperature and stirred for 4 hr.

The crude reaction contents are transferred to a 100 mL round bottom flask for distillation. Methylene chloride solvent is removed at RT under reduced pressure (20 torr (2.7 kPa)) to leave mixture (14.2 g) of product and N,N-diethyl-2,3,3,3-tetrafluoropropanamide (DETFP). This mixture is slowly poured over 10 mL of cooled 10% aqueous HCl solution. No exotherm was observed during quenching. After this mixture has warmed to room temperature, the organic phase is removed and set aside. The aqueous phase is extracted with three times with 10 mL hexane. The organic phase is combined with the hexane phases and further washed with deionized water (3×2 mL). The organic and hexane phases are combined and dried over MgSO₄ and concentrated in vacuo to afford 7.4 g of material which is characterized by ¹⁹F and ¹H NMR, and GC/MS.

### Product Analysis and Mass Balance:

Of the 7.4 g isolated material:
30 wt. % (2.2 g) is the desired product: methyl 2-fluoro-2-phenylacetate.
65 wt. % (4.8 g) is residual byproduct amide (DETFP).
5 wt. % (0.4 g) is methyl mandelate dimer product.
   Of the 9.1 g expected DETFP:
47 wt. % (4.3 g) was removed during water washes.
53 wt. % (4.8 g) stayed with the product
   Of the 5.2 g expected methyl 2-fluoro-2-phenylacetate product:
42 wt. % (2.2 g) was recovered.
58 wt. % (3.0 g) was lost during water washes.

Unlike 1,1,2,2-tetrafluoroethyl-N,N-dimethylamine, 1,1,2,3,3,3-hexafluoropropyl-N,N-diethyl amine gives the fluorinated product in low yield (30%) accompanied by substantial amounts of byproduct amide, despite repeated washing and extraction. As a result, the byproduct amide also is poorly recovered.

### Example 3

### Conversion of N,N-dimethyl difluoroacetamide to 1,1,2,2-Tetrafluoroethyl-N,N-dimethylamine

N,N-Dimethyl difluoroacetamide (4.0 g, 32.5 mmol) recovered from the reaction described in Example 1 and dried with molecular sieves, is combined with COF₂ (2.6 g, 40 mmol) in a 25 cc volume pressure vessel (shaker tube) and shaken at about room temperature (20-25° C.) for 16 hr. The shaker tube is vented and 4.5 g of liquid is recovered, avoiding exposure to water, such as moist atmosphere. Distillation in a simple microdistillation apparatus gives 4.0 g of 1,1,2,2-tetrafluoroethyl-N,N-dimethylamine, 85% yield based on the starting 4.0 g of N,N-dimethyl difluoroacetamide.

What is claimed is:

1. Process for making a fluorinated product comprising:
contacting an alcohol with 1,1,2,2-tetrafluoroethyl-N,N-dimethylamine to produce a product mixture containing the fluorinated product and N,N-dimethyl difluoroacetamide;
quenching the product mixture in water to form a first organic phase and an aqueous phase;
recovering fluorinated product by separating said first organic phase from said aqueous phase;
treating the aqueous phase to recover N,N-dimethyl difluoroacetamide; and
converting recovered N,N-dimethyl difluoroacetamide to 1,1,2,2-tetrafluoroethyl-N,N-dimethylamine.

2. The process of claim 1 further including extracting said aqueous phase with liquid hydrocarbon selected from the group consisting of alkane or aromatic to form a second organic phase and an extracted aqueous phase, combining said second organic phase with said first organic phase and evaporating said liquid hydrocarbon from the combined organic phase to recover the fluorinated compound.

3. The process of claim 2 further including washing said combined organic phase with water before said evaporating.

4. The process of claim 1 wherein said contacting of an alcohol with 1,1,2,2-tetrafluoroethyl-N,N-dimethylamine is performed in the presence of a halogenic or ether solvent.

5. The process of claim 1 when wherein said quenching is performed with water containing acid.

6. The process of claim 1 wherein said treating the aqueous phase to recover N,N-dimethyl difluoroacetamide comprises neutralizing said aqueous phase.

7. The process of claim 1 wherein said treating the aqueous phase to recover N,N-dimethyl difluoroacetamide comprises separating N,N-dimethyl difluoroacetamide from the aqueous phase by distillation.

8. The process of claim 1 wherein said converting recovered N,N-dimethyl difluoroacetamide to 1,1,2,2-tetrafluoroethyl-N,N-dimethylamine is performed by contacting with carbonyl fluoride.

9. The process of claim 1 wherein said converting recovered N,N-dimethyl difluoroacetamide to 1,1,2,2-tetrafluoroethyl-N,N-dimethylamine is performed by contacting with a chlorinating agent to produce a dichloride and contacting the dichloride with an alkali metal fluoride or tetraalkylammonium fluoride.
